(19) United States
(12) Patent Application Publication
(10) Pub. No.: US 2009/0142430 A1
(43) Pub. Date: Jun. 4, 2009

(54) Methods for Preventing or Treating Complications of Airway Control Devices

(76) Inventors: Ira Sanders, North Bergen, NJ (US); Christopher Shaari, Demarest, NJ (US)

Correspondence Address: PATENT DOCKET ADMINISTRATOR;LOWENSTEIN SANDLER PC, 65 LIVINGSTON AVENUE, ROSELAND, NJ, 07068, US

(21) Appl. No.: 12/165,847

(22) Filed: Jul. 1, 2008

Related U.S. Application Data
(60) Provisional application No. 60/992,278, filed on Dec. 4, 2007.

(51) Int. Cl. A61K 35/74; A61M 1/00

(52) U.S. Cl. 424/780; 604/514

(57) ABSTRACT

Disclosed in certain embodiments is a method of treating or preventing complications of airway control devices comprising administering to a patient having an airway control device a pharmaceutical composition comprising botulinum neurotoxin to one or more of the upper or lower aerodigestive secretory glands, the cricopharyngeus or the gastric or esophageal mucosal wall of the patient.

This application claims priority to U.S. Provisional Ser. No. 60/992,278 which is hereby incorporated by reference.

### FIELD OF THE INVENTION

This invention relates to compositions and methods for reducing or preventing complications of airway control devices such as endotracheal tubes.

### BACKGROUND

Patients sometimes require insertion of devices into their breathing passages. Most often these devices are inserted when there is a need to use a ventilator to breathe for the patient. The most common of these devices is an endotracheal tube (ET). An ET passes from outside the patient through the nose or mouth and into the trachea, the main air passage of the lungs. During inspiration air is pumped through the ET into the lungs and expired air passes back through the ET.

If there is no seal around the end of the ET pressurized air would leak around the tube and out the mouth. A seal is normally made by a balloon that surrounds the end of the tube. These tubes are called cuffed ETs. When inflated, the balloon contacts the tracheal wall and forms a seal so that pressurized air goes into the lungs. However, there is a limit to how strong this seal can be made. Specifically, if balloon pressure on the mucosa prevents blood flow to the mucosa it will cause damage to the tissue. As a result balloon pressure is usually limited to 25 cm H₂O.

A second reason for a balloon seal is to prevent fluids from seeping down from the mouth into the lungs. Normally the larynx and the cough reflexes of the lung prevent saliva, food or gastric contents from entering the lungs. However, intubated patients are often unconscious or too weak to cough. Moreover, the ET keeps a direct mechanical conduit open into the lungs thereby neutralizing most airway defenses. Therefore, patients who are intubated and ventilated cannot protect themselves from these fluids. As these fluids contain bacteria they can rapidly cause pneumonia.

### Ventilator Associated Pneumonia

The pneumonia arising during intubation and ventilation is called ventilator associated pneumonia (VAP). VAP mostly afflicts patients in intensive care units. These patients often have other serious medical problems impairing their ability to resist pneumonia. Moreover, ICUs have very virulent bacteria in the environment. Lastly, the patients remain on the ventilator with all the impaired airway defenses that made them susceptible to the pneumonia. For these reasons VAP has a very high mortality rate ranging from 20% to 50%. These clinically significant infections prolong duration of mechanical ventilation and ICU length of stay, underscoring the financial burden these infections impose on the health care system¹.

The gastrointestinal tract is thought to play an important role in the pathogenesis of VAP because the stomach often becomes colonized with Gram negative bacteria during critical illness, and enteric Gram-negative organisms are the most frequent microorganisms isolated from culture in patients with VAP. Known as the “gastropulmonary hypothesis”, VAP is thought to occur through the following sequence: The stomach is colonized from either endogenous or exogenous sources, followed by retrograde colonization of the oropharynx. Finally, the lower respiratory tract is colonized from sustained leakage of contaminated secretions around the cuff of the ET.

See, e.g., Davis Kimberly A, Ventilator Associated Pneumonia: A Review. J Intensive Care Med 2006, 21; 211; WO06078998A2: METHODS AND COMPOSITIONS FOR DECREASING SALIVA PRODUCTION; Dressler, Dirk and Benecke, Reiner (2007) ‘Pharmacology of therapeutic botulinum toxin preparations’, Disability & Rehabilitation, 29:23, 1761-1768.

In young children the diameter of the airway is so narrow that there is no space for a balloon cuff. Therefore an uncuffed ET is used whose outside diameter is large enough to contact the tracheal wall. This forms the necessary seal but is undesirable as the tube wall is usually made of a stiff material, in order to keep the airway open. This material is much harder then a flexible balloon, with the result that it can damage the delicate tracheal mucosa. This is often accelerated by the mechanical effect of ventilation, as the movement of the tube against mucosa causes erosion and ulceration. In children these erosions often get infected. Infections rapidly spread around the trachea. As the trachea swells when infected it can cause a narrowing of the airway to the point of obstruction.

Even when acute airway problems are avoided the damaged tracheal wall can scar. Over weeks to months the scar constricts inward eventually narrowing the airway to the point that the patient can't breathe. This complication is called tracheal stenosis and can be lethal. Treatment of tracheal stenosis is difficult and the patient may need to have a tracheostomy tube placed or extensive surgery to maintain an airway.

Recently, ETs have been introduced that have suction mechanisms integrated in the tube. These tubes are designed to suction fluids from beneath the balloon before they can reach the lungs. Published studies generally support the thinking that preventing these fluids from reaching the lungs will prevent pneumonia. However, these devices can injure mucosa or can only be used intermittently.

Newer devices for maintaining the airway, such as laryngeal masks, avoid the need to place an ET. However, the seal of a laryngeal mask is not adequate for positive pressure ventilation and cannot prevent saliva leaking around it.

### Prevention of Pneumonia

While VAP is pneumonia acquired as a result of intubations, sometimes unrecognized pneumonias cause a patient to require intubation. If the patient acquires the pneumonia within a health care facility it usually is due to virulent and potentially multi-drug resistant bacteria that can colonize those facilities. These pneumonias are termed hospital acquired pneumonias (HAP) and those acquired outside the hospital are community acquired pneumonia (CAP). This invention encompasses the prevention of HAP and CAP. As some conditions are known to have a high incidence of pneumonia or respiratory problems severe enough to require ventilation these conditions are encompassed by this invention. These conditions include but are not limited to bronchitis, brochiectasis, congestive heart failure, any condition that may impair consciousness, emphysema, asthma, interstitial lung fibrosis, renal failure, severe hypertension, history of myocardial infarction or cerebrovascular incident.

### Anti-Cholinergic Drugs²

During short durations of intubation, such as during surgery, anesthesiologists administer systemic drugs that block saliva production. These drugs block the nerve messages to the salivary glands that cause saliva production. Anti-cholinergic agents useful in the methods and compositions described herein are tertiary and quaternary amine anti-cholinergic agents such as tropicamide, glycopyrrolate, cyclopentolate, atropine, hyoscyamine, scopolamine, hyoscine, eucatropine, homatropine, benzhexol, benztropine, apoatropine, propantheline, pirenzepine, ipratropium, methylatropine, homatropine methylbromide, biperiden, procyclidine, a salt thereof, and combinations thereof. More preferably, an anti-cholinergic agent useful in the methods and compositions described herein is tropicamide, cyclopentolate, or glycopyrrolate. The most commonly used drug is glycopyrolate. Although these drugs are helpful during the relative short durations needed for surgical procedures, they have side effects during chronic use. They reduce the body's sweating ability, can even cause fever and heat stroke in high temperatures. Dry mouth, difficult urinating, headaches, diarrhea, constipation, blurred vision and drowsiness are all observed side effects of the drug.

### Botulinum Toxin

The anaerobic, gram positive bacterium Clostridium botulinum produces a potent polypeptide neurotoxin, botulinum toxin, which causes a neuroparalytic illness in humans and animals referred to as botulism. The spores of Clostridium botulinum are found in soil and can grow in improperly sterilized and sealed food containers of home based canneries, which are the cause of many of the cases of botulism. The effects of botulism typically appear 18 to 36 hours after eating the foodstuffs infected with a Clostridium botulinum culture or spores. The botulinum toxin can apparently pass unattenuated through the lining of the gut and attack peripheral motor neurons. Symptoms of botulinum toxin intoxication can progress from difficulty walking, swallowing, and speaking to paralysis of the respiratory muscles and death. Botulinum toxin type A is the most lethal natural biological agent known to man. About 50 picograms of botulinum toxin (purified neurotoxin complex) type A is a LD50 in mice. One unit (U) of botulinum toxin is defined as the LD50 upon intraperitoneal injection into female Swiss Webster mice weighing 18-20 grams each. Seven immunologically distinct botulinum neurotoxins have been characterized, these being respectively botulinum neurotoxin serotypes A, B, C1, D, E, F and G each of which is distinguished by neutralization with type-specific antibodies. The different serotypes of botulinum toxin vary in the animal species that they affect and in the severity and duration of the paralysis they evoke. For example, it has been determined that botulinum toxin type A is 500 times more potent, as measured by the rate of paralysis produced in the rat, than is botulinum toxin type B. Additionally, botulinum toxin type B has been determined to be non-toxic in primates at a dose of 480 U/kg which is about 12 times the primate LD50 for botulinum toxin type A. Botulinum toxin apparently binds with high affinity to cholinergic motor neurons, is translocated into the neuron and blocks the release of acetylcholine. Botulinum toxins have been used in clinical settings for the treatment of neuromuscular disorders characterized by hyperactive skeletal muscles. Botulinum toxin type A has been approved by the U.S. Food and Drug Administration for the treatment of blepharospasm, strabismus and hemifacial spasm. Non-type A botulinum toxin serotypes apparently have a lower potency and/or a shorter duration of activity as compared to botulinum toxin type A. Clinical effects of peripheral intramuscular botulinum toxin type A are usually seen within one week of injection. The typical duration of symptomatic relief from a single intramuscular injection of botulinum toxin type A averages about three months. Although all the botulinum toxins serotypes apparently inhibit release of the neurotransmitter acetylcholine at the neuromuscular junction, they do so by affecting different neurosecretory proteins and/or cleaving these proteins at different sites. For example, botulinum types A and E both cleave the 25 kiloDalton (kD) synaptosomal associated protein (SNAP-25), but they target different amino acid sequences within this protein. Botulinum toxin types B, D, F and G act on vesicle-associated protein (VAMP, also called synaptobrevin), with each serotype cleaving the protein at a different site. Finally, botulinum toxin type C1 has been shown to cleave both syntaxin and SNAP-25. These differences in mechanism of action may affect the relative potency and/or duration of action of the various botulinum toxin serotypes. Significantly, it is known that the cytosol of pancreatic islet B cells contains at least SNAP-25 (Biochem J 1; 339 (Pt 1): 159-65 (April 1999)), and synaptobrevin (Mov Disord 1995 May; 10(3): 376). With regard to the use of a botulinum toxin to treat a pancreatic related disorder, it is known to treat a form of pancreatitis by injecting a botulinum toxin into the minor duodenal papilla (because of the proximity of the minor papilla to the pancreatic duct) to thereby relax a constricted pancreatic duct (pancreatic divisum) and increase the flow of pancreatic juice through the pancreatic duct into the duodenum. Gastrointest Endosc 1999 October; 50 (4): 545-548. The molecular weight of the botulinum toxin protein molecule, for all seven of the known botulinum toxin serotypes, is about 150 kD. Interestingly, the botulinum toxins are released by Clostridial bacterium as complexes comprising the 150 kD botulinum toxin protein molecule along with associated non-toxin proteins. Thus, the botulinum toxin type A complex can be produced by Clostridial bacterium as 900 kD, 500 kD and 300 kD forms. Botulinum toxin types B and C1 is apparently produced as only a 500 kD complex. Botulinum toxin type D is produced as both 300 kD and 500 kD complexes. Finally, botulinum toxin types E and F are produced as only approximately 300 kD complexes. The complexes (i.e. molecular weight greater than about 150 kD) are believed to contain a non-toxin hemaglutinin protein and a non-toxin and non-toxic nonhemaglutinin protein. These two non-toxin proteins (which along with the botulinum toxin molecule comprise the relevant neurotoxin complex) may act to provide stability against denaturation to the botulinum toxin molecule and protection against digestive acids when toxin is ingested. Additionally, it is possible that the larger (greater than about 150 kD molecular weight) botulinum toxin complexes may result in a slower rate of diffusion of the botulinum toxin away from a site of intramuscular injection of a botulinum toxin complex. In vitro studies have indicated that botulinum toxin inhibits potassium cation induced release of both acetylcholine and norepinephrine from primary cell cultures of brainstem tissue. Additionally, it has been reported that botulinum toxin inhibits the evoked release of both glycine and glutamate in primary cultures of spinal cord neurons and that in brain synaptosome preparations botulinum toxin inhibits the release of each of the neurotransmitters acetylcholine, dopamine, norepinephrine, CGRP and glutamate. Botulinum toxin type A can be obtained by establishing and growing cultures of Clostridium botulinum in a fermenter and then harvesting and purifying the fermented mixture in accordance with known procedures. All the botulinum toxin serotypes are initially synthesized as inactive single chain proteins which must be cleaved or nicked by proteases to become neuroactive. The bacterial strains that make botulinum toxin serotypes A and G possess endogenous proteases and serotypes A and G can therefore be recovered from bacterial cultures in predominantly their active form. In contrast, botulinum toxin serotypes C1, D and E are synthesized by nonproteolytic strains and are therefore typically unactivated when recovered from culture. Serotypes B and F are produced by both proteolytic and nonproteolytic strains and therefore can be recovered in either the active or inactive form. However, even the proteolytic strains that produce, for example, the botulinum toxin type B serotype only cleave a portion of the toxin produced. The exact proportion of nicked to unnicked molecules depends on the length of incubation and the temperature of the culture. Therefore, a certain percentage of any preparation of, for example, the botulinum toxin type B toxin is likely to be inactive, possibly accounting for the known significantly lower potency of botulinum toxin type B as compared to botulinum toxin type A. The presence of inactive botulinum toxin molecules in a clinical preparation will contribute to the overall protein load of the preparation, which has been linked to increased antigenicity, without contributing to its clinical efficacy. Additionally, it is known that botulinum toxin type B has, upon intramuscular injection, a shorter duration of activity and is also less potent than botulinum toxin type A at the same dose level. It has been reported that botulinum toxin type A has been used in clinical settings as follows:

(1) about 75-125 units of BOTOX®1 per intramuscular injection (multiple muscles) to treat cervical dystonia;
(2) 5-10 units of BOTOX® per intramuscular injection to treat glabellar lines (brow furrows) (5 units injected intramuscularly into the procerus muscle and 10 units injected intramuscularly into each corrugator supercilii muscle);
(3) about 30-80 units of BOTOX® to treat constipation by intrasphincter injection of the puborectalis muscle;
(4) about 1-5 units per muscle of intramuscularly injected BOTOX® to treat blepharospasm by injecting the lateral pre-tarsal orbicularis oculi muscle of the upper lid and the lateral pre-tarsal orbicularis oculi of the lower lid.
(5) to treat strabismus, extraocular muscles have been injected intramuscularly with between about 1-5 units of BOTOX®, the amount injected varying based upon both the size of the muscle to be injected and the extent of muscle paralysis desired (i.e. amount of diopter correction desired).
(6) to treat upper limb spasticity following stroke by intramuscular injections of BOTOX® into five different upper limb flexor muscles, as follows:
(a) flexor digitorum profundus: 7.5 U to 30 U
(b) flexor digitorum sublimus: 7.5 U to 30 U
(c) flexor carpi ulnaris: 10 U to 40 U
(d) flexor carpi radialis: 15 U to 60 U
(e) biceps brachii: 50 U to 200 U. Each of the five indicated muscles has been injected at the same treatment session, so that the patient receives from 90 U to 360 U of upper limb flexor muscle BOTOX® by intramuscular injection at each treatment session.

BOTOX® is available from Allergan, Inc., of Irvine, Calif. under the tradename BOTOX®. The success of botulinum toxin type A to treat a variety of clinical conditions has led to interest in other botulinum toxin serotypes. A study of two commercially available botulinum type A preparations (BOTOX® and Dysport®) and preparations of botulinum toxins type B and F (both obtained from Wako Chemicals, Japan) has been carried out to determine local muscle weakening efficacy, safety and antigenic potential. Botulinum toxin preparations were injected into the head of the right gastrocnemius muscle (0.5 to 200.0 units/kg) and muscle weakness was assessed using the mouse digit abduction scoring assay (DAS). ED50 values were calculated from dose response curves. Additional mice were given intramuscular injections to determine LD50 doses. The therapeutic index was calculated as LD50/ED50. Separate groups of mice received hind limb injections of BOTOX® (5.0 to 10.0 units/kg) or botulinum toxin type B (50.0 to 400.0 units/kg), and were tested for muscle weakness and increased water consumption, the later being a putative model for dry mouth. Antigenic potential was assessed by monthly intramuscular injections in rabbits (1.5 or 6.5 ng/kg for botulinum toxin type B or 0.15 ng/kg for BOTOX®). Peak muscle weakness and duration were dose related for all serotypes. DAS ED50 values (units/kg) were as follows: BOTOX®: 6.7, Dysport®: 24.7, botulinum toxin type B: 27.0 to 244.0, botulinum toxin type F: 4.3. BOTOX® had a longer duration of action than botulinum toxin type B or botulinum toxin type F. Therapeutic index values were as follows: BOTOX®: 10.5, Dysport®: 6.3, botulinum toxin type B: 3.2. Water consumption was greater in mice injected with botulinum toxin type B than with BOTOX®, although botulinum toxin type B was less effective at weakening muscles. After four months of injections 2 of 4 (where treated with 1.5 ng/kg) and 4 of 4 (where treated with 6.5 ng/kg) rabbits developed antibodies against botulinum toxin type B. In a separate study, 0 of 9 BOTOX® treated rabbits demonstrated antibodies against botulinum toxin type A. DAS results indicate relative peak potencies of botulinum toxin type A being equal to botulinum toxin type F, and botulinum toxin type F being greater than botulinum toxin type B. With regard to duration of effect, botulinum toxin type A was greater than botulinum toxin type B, and botulinum toxin type B duration of effect was greater than botulinum toxin type F. As shown by the therapeutic index values, the two commercial preparations of botulinum toxin type A (BOTOX® and Dysport®) are different. The increased water consumption behavior observed following hind limb injection of botulinum toxin type B indicates that clinically significant amounts of this serotype entered the murine systemic circulation. The results also indicate that in order to achieve efficacy comparable to botulinum toxin type A, it is necessary to increase doses of the other serotypes examined. Increased dosage can comprise safety. Furthermore, in rabbits, type B was more antigenic than was BOTOX®, possibly because of the higher protein load injected to achieve an effective dose of botulinum toxin type B.

### Acetylcholine

Typically only a single type of small molecule neurotransmitter is released by each type of neuron in the mammalian nervous system. The neurotransmitter acetylcholine is secreted by neurons in many areas of the brain, but specifically by the large pyramidal cells of the motor cortex, by several different neurons in the basal ganglia, by the motor neurons that innervate the skeletal muscles, by the preganglionic neurons of the autonomic nervous system (both sympathetic and parasympathetic), by the postganglionic neurons of the parasympathetic nervous system, and by some of the postganglionic neurons of the sympathetic nervous system. Essentially, only the postganglionic sympathetic nerve fibers to the sweat glands, the piloerector muscles and a few blood vessels are cholinergic and most of the postganglionic neurons of the sympathetic nervous system secret the neurotransmitter norepinephine. In most instances acetylcholine has an excitatory effect. However, acetylcholine is known to have inhibitory effects at some of the peripheral parasympathetic nerve endings, such as inhibition of the heart by the vagal nerve. The efferent signals of the autonomic nervous system are transmitted to the body through either the sympathetic nervous system or the parasympathetic nervous system. The preganglionic neurons of the sympathetic nervous system extend from preganglionic sympathetic neuron cell bodies located in the intermediolateral horn of the spinal cord. The preganglionic sympathetic nerve fibers, extending from the cell body, synapse with postganglionic neurons located in either a paravertebral sympathetic ganglion or in a prevertebral ganglion. Since, the preganglionic neurons of both the sympathetic and parasympathetic nervous system are cholinergic, application of acetylcholine to the ganglia will excite both sympathetic and parasympathetic postganglionic neurons. Acetylcholine activates two types of receptors, muscarinic and nicotinic receptors. The muscarinic receptors are found in all effector cells stimulated by the postganglionic neurons of the parasympathetic nervous system, as well as in those stimulated by the postganglionic cholinergic neurons of the sympathetic nervous system. The nicotinic receptors are found in the synapses between the preganglionic and postganglionic neurons of both the sympathetic and parasympathetic. The nicotinic receptors are also present in many membranes of skeletal muscle fibers at the neuromuscular junction.

### Botulinum Toxin and Saliva

U.S. Pat. No. 5,766,605 discloses a method of treating sialorrhea with botulinum toxin by needle injection of the salivary glands or the ganglia innervated the glands. Injections of botulinum toxin into salivary tissue has been performed for years to treat drooling associated with neurologic aliments such as Parkinson's disease or cerebral palsy. There are over 100 published clinical studies that nearly uniformly demonstrate that a botulinum toxin injection into the salivary glands is a safe and effective therapy for reducing sialorrhea or drooling. Studies using botulinum Type A (Allergan) or Type B (Solstice Neuroscience), have demonstrated effectiveness in controlling drooling.

VAP is Distinct from Drooling

VAP is a distinct problem from drooling. Drooling is usually cause by excessive or normal production of saliva that cannot be properly swallowed and then leaks out the front of the mouth. VAP occurs when the normal or even diminished production of saliva seeps around an ET and into the airway, thereby becoming problematic. In the several years of published studies on botulinum toxin therapy for salivation, not one study has investigated the non-obvious problem of salivary contamination of the lungs and its contribution to VAP.

Moreover, the situation in VAP presents unique challenges and opportunities for therapy.

The patient is usually unconscious and therefore there is no gag reflex and little movement in the oral cavity. This allows topical application in the oral in ways that would never be tolerated by a conscious patient.

The unconsciousness of the patient as well as the physical presence of the ET or other airway device neutralizes all the airway defenses of the patient.

The complications experienced by a ventilated patient are very serious and result in high rates of morbidity and mortality.

The patient is not eating so there is no need for saliva.

Other secretions become relatively more important, specifically saliva produced by minor salivary glands, and nasal, pharyngeal, laryngeal and pulmonary respiratory mucosal glands.

### SUMMARY OF THE INVENTION

Disclosed are methods of preventing or treating complications of airway control or airway disorders in a mammal. These methods involve applying botulinum toxin or its equivalent, alone or in combination with other drugs or devices, thereby minimizing, preventing, treating, or enabling easier management of the problems in the patient.

The botulinum toxin can be one or more of the serotypes A, B, C, D, E, F, or G and can be modified, a chimera, hybrid, recombinant or altered but retains the same biological effects as wild type botulinum toxin. The dose of the botulinum toxin can be, e.g., in an amount of between 0.01 units and 5000 units, such as between 0.01 unit and 500 units.

In certain embodiments, the invention is directed to method of treating or preventing complications of airway control devices and ventilation comprising administering to a patient having an airway control device a pharmaceutical composition comprising botulinum neurotoxin to one or more of the upper or lower aerodigestive secretory glands, the cricopharyngeus or the gastric or esophageal mucosal wall of the patient.

In certain embodiments, the complication is ventilator associated pneumonia.

In certain embodiments, the airway control device is an endotracheal tube, a tracheostomy tube or a laryngeal mask. The endotracheal tube can optionally have subglottic suction capability or high volume low pressure cuffs

In certain embodiments, the pharmaceutical composition further comprises complexing proteins and optional pharmaceutically acceptable excipients.

In certain embodiments, botulinum neurotoxin is administered to the secretory glands by needle injection, needleless injection or topical application.

In certain embodiments, the secretory glands are salivary glands.

In certain embodiments, the secretory glands are one or more parotid, submaxillary, sublingual, or mucosal or submucosal glands.

In certain embodiments, then mucosal glands are one or more oral cavity, pharyngeal, nasal, sinus, laryngeal, tracheal or bronchial, or esophageal or gastric mucosal glands.

In certain embodiments, the pharmaceutical composition is administered at the time of the intubation of the airway control device.

In certain embodiments, the pharmaceutical composition is administered prior to securing or introducing the airway control device. For example, 1 second to 6 months prior, 1 day to 2 months prior or 1 week to 1 month prior. In other embodiments, the administration can be from 30 minutes prior to 24 hours prior to securing or introducing the airway control device, e.g., 1 or 2 hours prior to securing or introducing the airway control device. The present invention is also directed to methods of administering more than one dose according to a dosing regimen.

In certain embodiments, the pharmaceutical composition is administered after securing or introducing the airway control device. For example, 1 second to 6 months after, 1 day to 2 months after or 1 week to 1 month after. In other embodiments, the administration can be from 30 minutes after to 24 hours after securing or introducing the airway control device, e.g., 1 or 2 hours after securing or introducing the airway control device. The present invention is also directed to methods of administering more than one dose according to a dosing regimen.

In other embodiments, the administration include administration of at least two or all three of before, during and after securing or introducing the airway control device

In certain embodiments, the methods of the present invention further comprise administering a second salivation reducing agent such as an anticholinergic agent (e.g., atropine, iatropium and/or glycopyrolate). The invention is also directed to pharmaceutical composition comprising botulinum neurotoxin and a second salivation agent as well as complexing proteins and other optional excipients.

In certain embodiments, the methods of the present invention further comprise utilizing other medical procedures such as administering an antacid, raising the head, manually suctioning trachea and or oral secretions or orally rinsing with antiseptics.

The present invention is also directed to a method of treating or preventing complications associated with pulmonary disease comprising administering to a patient having a pulmonary disease a pharmaceutical composition comprising botulinum neurotoxin to one or more of the upper or lower aerodigestive airway secretory glands, the cricopharyngeus or the esophageal or gastric mucosal wall of the patient. The pulmonary disease can be, e.g., bronchitis, COPD, asthma or a neurological disease causing dysphagia (e.g., Parkinson's disease, Alzheimers, cerebral palsy, myasthenia gravis, amyotrophic lateral sclerosis, head trauma or stroke). The present invention can also be utilized in patients who are being intubated for surgery, particularly surgery that is known to need post operative ventilation such as cardiothoracic procedure.

### DETAILED DESCRIPTION

Botulinum toxin (BT) means the wild type neurotoxin isolated and purified from Clostridia botulinum, butyricum, or beratti. These include but are not limited by the recognized serotypes A, B, C, D, E, F, and G.

Also included within the definition of BT are other entities that have the same biological activity in blocking neurotransmitter release within neurons. These toxins include without limitation chimeras, hybrids, modified, or altered or modified wild type botulinum toxin. Also included is tetanus toxin.

Therapeutic preparations of botulinum toxin (BT) consist of botulinum neurotoxin (BNT), complexing proteins and excipients. Depending on the target tissue BT can block the cholinergic neuromuscular or the cholinergic autonomic innervation of exocrine glands and smooth muscles. Additional effects can be demonstrated on the muscle spindle organ. Indirect effects on the central nervous system are numerous, direct ones have not been recorded after intramuscular injections.

BT type A is being distributed as Botox (Allergan Inc), Dysport (Ipsen Inc) and Xeomin (Merz Pharmaceuticals), BT type B as NeuroBloc/Myobloc (Solstice Neuroscience). Adverse effects can be obligate, local or systemic. The adverse effect profiles of the available BT preparations are similar. BT type B, however, has additional systemic autonomic adverse effects. Long-term treatment does not produce additive adverse effects. BNT can be partially or completely blocked by antibodies. The major risk factors for antibody-induced therapy failure are the amount of BNT applied at each injection series, the interval between injection series and the specific biological activity (SBA) of the BT preparation used.

### The SBA is

### 5 for NeuroBloc,

### 60 for Botox,

### 100 for Dysport and

167 MUE/ng BNT for Xeomin (MU-E: equivalence mouse units)³.

BT can be delivered to the secretory glands by needle injection, needleless jet injection, topical application, topical spray, aerosols, nebulizers or other methods known in the art. BT can be applied within or near: the gland, the ducts draining the gland, or to the parasympathetic ganglia whose nerves innervate the gland.

The secretory glands include but are not limited to major and minor salivary glands and respiratory secretory and mucus glands in the nasal cavity, pharynx, larynx, trachea and bronchus.

The toxin can be presented as a sterile pyrogen-free aqueous solution or dispersion and as a sterile powder for reconstitution into a sterile solution or dispersion.

Optionally, a tonicity adjusting agents such as sodium chloride, glycerol and/or various sugars can be added. Stabilizers such as human serum albumin may also be included. The formulation may optionally be preserved by means of a suitable pharmaceutically acceptable preservative such as a paraben.

In certain embodiments, the toxin is formulated in a unit dosage form, e.g., as a sterile solution in a vial or as a vial or sachet containing a lyophilized powder for reconstituting a suitable vehicle such as saline for injection.

In one embodiment, the Botulinum toxin is formulated in a solution containing saline and pasteurized human serum albumin, which stabilizes the toxin and minimizes loss through non-specific adsorption. The solution is sterile filtered (0.2 micron filter), filled into individual vials and then vacuumdried to give a sterile lyophilized powder. In use, the powder can be reconstituted by the addition of sterile unpreserved normal saline (sodium chloride 0.9% for injection).

Medical conditions treated include but are not limited to any condition in which excess airway secretions are problematic, examples being intubated and tracheotomized patients, airway hygiene maintenance in chronic lung or neurological diseases, and patients with swallowing disorders.

Application of BT may be combined with other anticholinergic drugs (anti-AchE) to achieve a more rapid onset of salivary production blockage. Either BT or anti-AchE may both be applied to the target glands, however only anti-AchE can be given systemically (intramuscular or intravenous). The relative timing of application can vary: Anti-AchE can be given concurrently or 1 week before or after BT. Repeated doses can be given to titrate the effects on secretions.

Application of BT to the airway may be combined with application to the cricopharyngeus muscle. Relaxation of the cricopharyngeus muscles decreases resistance to salivary drainage and in ambulatory patients with dysphagia it aids in swallowing.

### SPECIFIC EXAMPLES

### #1. Botulinum Toxin A Injection after Intubation (Prophetic)

A 50 year old patient is intubated for pulmonary edema and lung cancer, and it is anticipated that the endotracheal tube will be in place for more than 48 hours. Within 1 hour after intubation the submandibular and parotid salivary glands are palpated and injected with 25 units of type A botulinum toxin for a total of 100 units. The patient's normal production of saliva is reduced. The frequency with which the nurse must suction the salivary secretions from his throat and lungs is reduced, and the patient's risk for VAP is reduced. The overall length of ICU stay is reduced as well since he did not develop VAP. The patient was more comfortable as well since fewer episodes of endotracheal suctioning were required.

This example shows prevention of VAP by needle injection of BT-A to major salivary glands.

### #2. Botulinum Toxin B Injection after Intubation (Prophetic)

A 50 year old patient is intubated for pulmonary edema and lung cancer, and it is anticipated that the endotracheal tube will be in place for more than 48 hours. The submandibular and parotid salivary glands are palpated and injected with a total of 2500 units of type B botulinum toxin for a total of 10,000 units. The patient's normal production of saliva is reduced. The frequency with which the nurse must suction the salivary secretions from his throat and lungs is reduced, and the patient's risk for VAP is reduced. The overall length of ICU stay is reduced as well since he did not develop VAP. The patient was more comfortable as well since fewer episodes of endotracheal suctioning were required.

This example shows prevention of VAP by needle injection of BT-B to major salivary glands.

### #3. Botulinum Toxin A Injection before Intubation (Prophetic)

A 50 year old man is scheduled for cardiothoracic surgery. As this surgery is usually followed by intubation and ventilation with a high risk of VAP he is given prophylactic injections of BT 2 days prior to the procedure. Injections are given as described in example #1.

This example shows prevention of VAP by prophylactic injection prior to intubation.

### #4. Botulinum Toxin A Topical Application (Prophetic)

Immediately after airway control a 3×1 inch gauze soaked in 1 cc solution of normal saline with 200 units BT-A. The gauze is draped under the tongue covering the undersurface of the tongue as well the mucosa of the floor of the mouth. The gauze is removed in 2 hours. Salivary production from the submandibular and sublingual glands decreases in 2 days to 20% of normal.

This example shows topical application of BT to a large surface area, specifically the undersurface of the tongue and floor of the mouth. The ducts from the sublingual glands exit beneath the tongue, while those of the submaxillary gland exit at Wharton's duct which is near the front of the floor of mouth.

### #5. Botulinum Toxin A and Anti-Cholinergic Drug Combination (Prophetic)

A 30 year old male is brought into the emergency room unconscious after a motorcycle accident and presumed head trauma. The patient is breathing spontaneously. The treating physician places a laryngeal mask airway. The treating physician believes that the patient may recover consciousness within 24 hours and wants to avoid placement of an ET tube. However, there is always the possibility that the patient will deteriorate.

The physician injects 0.004 mg/kg glycopyrolate into a thigh muscle to get rapid onset of salivary blocking and then injects the salivary gland with BT-A by the method of example A. Salivation decreases markedly within 1 hour.

Alternatively the treating physician injects each of the submaxillary and parotid glands with 0.001 mg glycopyrolate together with or followed by BT-A as described in example #1.

### #6. Botulinum Toxin A Application to all Secretory Glands (Prophetic)

A 70 year old patient has been in a coma and maintained on a ventilator through a cuffed tracheostomy tube for 6 months following hypoxic brain injury. Every 4 months the patient undergoes the following regimen to eliminate as much upper airway secretion as possible:

Injection with BT-A as described in example #1.
Topical, 1″×12″ inch gauze soaked in BT-A is carefully packed into the hypopharynx, oropharynx and oral cavity and left for 1 hour.
Spray, 20 units of aerosolized BT-A is sprayed into each nostril.
Spray, 20 units of aerosolized BT-A is sprayed into the trachea and bronchus through the tracheotomy after the tracheostomy tube is removed.

### #7. Botulinum Toxin A in Dysphagic Ambulatory Patient (Prophetic)

A 50 year old male with dysphagia and chronic bronchitis is at high risk of requiring intubation. His physician injects 20 units of BT-A into his cricopharyngeus muscle to aid in swallowing. He also passes a needle through the cricothyroid membrane and sprays 20 units mixed in 2 cc normal saline into the trachea. The BT-A drips down the walls of the trachea and into the bronchioles. In one week patient returns and reports improved swallowing with less coughing of mucous and less coughing during eating.

This example shows application of BT to decrease airway secretions and to allow easier drainage and swallowing of secretions, thereby avoiding spillover of secretions into the lungs.

### REFERENCES

1: Can J. Psychiatry. 2007 June; 52(6):377-84, “Clozapine-Induced Hypersalivation: A Review Of Treatment Strategies”, Sockalingam S, Shammi C, Remington G., Department of Psychiatry, University of Toronto, Ontario
2: Expert Rev Neurother. 2007 June; 7(6):637-47, “Botulinum Toxin In The Treatment Of Tremors, Dystonias, Sialorrhea And Other Symptoms Associated With Parkinson's Disease”, Sheffield J K, Jankovic J., Department of Neurology, Baylor College of Medicine, Parkinson's Disease Center & Movement Disorders Clinic
3: Ann Pharmacother. 2007 January; 41(1):79-85. Epub 2006 Dec. 26, “Botulinum Toxin A In The Treatment Of Sialorrhea”, Benson J, Daugherty K K., Kroger Pharmacy, Frankenmuth, Mich., USA.
4: Curr Opin Otolaryngol Head Neck Surg. 2006 December; 14(6):381-6, “Drooling”, Lal D, Hotaling A J, Department of Otolaryngology, Head and Neck Surgery, Loyola University Medical Center, Maywood, Ill. 60153, USA.
5: Oral Surg Oral Med Oral Pathol Oral Radiol Endod. 2006 January; 101(1):48-57, “Drooling Of Saliva: A Review Of The Etiology And Management Options”, Meningaud J P, Pitak-Arnnop P, Chikhani L, Bertrand J C, Department of Maxillofacial Surgery, Teaching Pitié-Salpêtrière Hospital, Paris.

We claim:

1. A method of treating or preventing complications of airway control devices comprising administering to a patient having an airway control device a pharmaceutical composition comprising botulinum neurotoxin to one or more of the upper or lower aerodigestive secretory glands, the cricopharyngeus or the gastric or esophageal mucosal wall of the patient.

2. The method of claim 1, wherein the complication is ventilator associated pneumonia.

3. The method of claim 1, wherein the airway control device is an endotracheal tube, a tracheostomy tube or a laryngeal mask.

4. The method of claim 1, wherein the pharmaceutical composition further comprises complexing proteins and optional pharmaceutically acceptable excipients.

5. The method of claim 1, wherein the botulinum neurotoxin is administered to the secretory glands by needle injection, needleless injection or topical application.

6. The method of claim 1, wherein the secretory glands are salivary glands.

7. The method of claim 1, wherein the secretory glands are one or more parotid, submaxillary, sublingual, or mucosal or submucosal glands.

8. The method of claim 7, wherein the mucosal glands are one or more oral cavity, pharyngeal, nasal, sinus, laryngeal, tracheal or bronchial, or esophageal or gastric mucosal glands.

9. The method of claim 1, wherein the botulinum neurotoxin is selected from the group consisting of serotypes A, B, C, D, E, F, G or a combination thereof.

10. The method of claim 1, wherein the botulinum neurotoxin is in a dose of between 0.01 units and 5000 units

11. The method of claim 1, wherein the pharmaceutical composition is administered at the time of the intubation of the airway control device.

12. The method of claim 1, wherein the pharmaceutical composition is administered prior to securing or introducing the airway control device.

13. The method of claim 1, wherein the pharmaceutical composition is administered after securing or introducing the airway control device.

14. The method of claim 1, further comprising administering a second salivation reducing agent.

15. The method of claim 14, wherein the second salivation reducing agent is an anticholinergic agent.

16. The method of claim 3, wherein the endotracheal tube has subglottic suction capability or high volume low pressure cuffs.

17. The method of claim 1, further comprising administering an antacid, raising the head, manually suctioning trachea and or oral secretions or orally rinsing with antiseptics.

18. A method of treating or preventing complications associated with pulmonary disease comprising administering to a patient having a pulmonary disease a pharmaceutical composition comprising botulinum neurotoxin to one or more of the upper or lower aerodigestive airway secretory glands, the cricopharyngeus or the esophageal or gastric mucosal wall of the patient.

19. The method of claim 18, wherein the pulmonary disease is bronchitis, COPD, asthma or a neurological disease causing dysphagia.

20. A pharmaceutical composition comprising botulinum neurotoxin and a second salivation inhibitor.
